(19) United States
(12) Patent Application Publication
(10) Pub. No.: US 2020/0043614 A1
(43) Pub. Date: Feb. 6, 2020

(54) TREATMENT COURSE DETERMINATION ASSISTANCE SYSTEM

(71) Applicants: Naoki WASHIDA, Narita-shi, Chiba (JP); Hiroshi ITOH, Tokyo (JP); Takumi ICHIKAWA, Tokyo (JP); Keigo SHIBAGAKI, Tokyo (JP); Taizan SUZUKI, Tokyo (JP); Manabu FUKUDA, Tokyo (JP); SOPHIA INTERNATIONAL CO., LTD., Tokyo (JP); CRI MIDDLEWARE CO., LTD., Tokyo (JP)

(72) Inventors: Naoki WASHIDA, Narita-shi (JP); Hiroshi ITOH, Tokyo (JP); Takumi ICHIKAWA, Tokyo (JP); Keigo SHIBAGAKI, Tokyo (JP); Taizan SUZUKI, Tokyo (JP); Manabu FUKUDA, Tokyo (JP)

(73) Assignees: Sophia International Co., Ltd., Tokyo (JP); CRI Middleware Co., Ltd., Tokyo (JP)

(21) Appl. No.: 15/735,691

(22) PCT Filed: Aug. 27, 2015
(86) PCT No.: PCT/JP2015/074272

§ 371 Date: Dec. 12, 2017

(30) Foreign Application Priority Data
Jun. 25, 2015 (JP) 2015-127439

(51) Int. Cl. G16H 50/70; G16H 50/20; G16H 80/00; G16H 10/60; H04L 12/18; H04L 12/58

(52) U.S. Cl. CPC G16H 50/70; G16H 50/20; H04L 51/38; G16H 10/60; H04L 12/1813; G16H 80/00

(57) ABSTRACT

A treatment course determination assistance system is configured to perform the steps of: allowing a review applicant to input details to be reviewed through use of an input unit; displaying, by a processing server, a designated reviewer candidate involved in treatment of the patient based on information read from the patient information database; allowing the review applicant to select a designated reviewer candidate desired to perform review based on professions of displayed designated reviewer candidates; selecting, by the processing server, one or more anonymous reviewer candidates extracted randomly based on professions and/or specialties relating to the details to be reviewed from the reviewer information database; and asking the designated reviewer candidates selected by the review applicant and the one or more anonymous reviewer candidates selected by the processing server whether or not to accept a request for cooperation on review.

### FIELD OF THE INVENTION

The present invention relates to a treatment course determination assistance system, which is configured to enable both opinions of a medical professional involved and an anonymous expert to be heard easily and further enable those who have had their opinions heard to review a treatment plan for a patient easily and express their opinions easily in decision of the treatment plan.

### DESCRIPTION OF THE RELATED ART

The medical industry has an aspect of difficulty in sharing information for treatment of a patient between a primary doctor and a home-visit nursing station. That is, it is difficult to share patient information and other related information in real time because information is mainly shared through delivery of a document or phone conversation, resulting in time lag in sharing information or a fear of interfering with daily business operations on the other side. In the first place, there is no choice but to rely on a physical medium, for example, a notebook, in many cases due to the lack of a system for sharing information on patients in a cross-sectional manner.

Further, there are actually many cases in which information sharing depends on an analog method because, even when the patient information is managed electronically, secondary use of data is extremely limited due to the lack of a common data standard between independent systems in a hospital to which the primary doctor belongs and the home-visit nursing station.

Under such circumstances, the patient has an opportunity to hear opinions of a third-party expert other than the primary doctor only through reception of a second opinion as an outpatient in the current situations. The reception of a second opinion requires reception of a patient referral document from the primary doctor, and in addition, the patient needs to find and visit another medical institution.

As a result, the patient currently has little opportunities to hear opinions of a third-party expert other than the primary doctor. Further, the primary doctor has his or her own specialty, and all the treatments necessary for the patient do not necessarily fail within the specialty. That is because treatments are sometimes difficult without covering different specialties of a plurality of doctors.

In such a case, in order to hear opinions of a third party expert other than the primary doctor, the patient first sees his or her primary doctor, receives a written related document, for example, a patient referral document, and visits another hospital with the written related document, for example, the patient referral document.

In Japanese Patent Application Laid-open No. 2011-39674, there is disclosed a system configured to allow the patient to use a terminal of a hospital to open a website screen provided for himself or herself, search for a hospital or a doctor from which or whom the patient wishes to receive a second opinion, and contact, by email, the hospital or other facilities from which the patient wishes to receive a second opinion.

The medical industry has hitherto been trying to allow a patient to receive a second opinion for a treatment plan of a primary doctor through use of a network. However, there has hitherto been no framework for allowing not only the patient but also the primary doctor and medical professionals involved such as a nurse, a nutritionist, a clinical engineer, and a social worker, who are already involved in the treatment, to hear opinions of other medical professionals involved and decide the treatment plan because information sharing means is not developed sufficiently as described above.

Further, there has been no prospect for a framework for easily asking an anonymous expert other than the medical professionals involved for an opinion under circumstances in which reception of a second opinion is difficult.

### SUMMARY OF INVENTION

Therefore, the present invention has an object to provide a treatment course determination assistance system, which is configured to enable both opinions of a medical professional involved and an anonymous expert to be heard easily and further enable those who have provided opinions to review a treatment plan for a patient easily and express their opinions easily in decision of the treatment plan.

In order to achieve the above-mentioned object, according to one embodiment of the present invention, there is provided a treatment course determination assistance system, which is configured to receive opinions from a plurality of reviewers in decision of a treatment plan for a patient, the treatment course determination assistance system including: a processing server configured to perform processing for supporting decision of the treatment plan; a patient information database having stored therein patient information capable of being read from the processing server; a reviewer information database having stored reviewer information capable of being read from the processing server; and an input/output device comprising an input unit configured to input information to the processing server and a display unit configured to output information from the processing server, in which the treatment course determination assistance system is configured to perform the steps of: allowing a review applicant to input information for identifying the patient and details to be reviewed for supporting decision of the treatment plan through use of the input unit; displaying, by the processing server, a designated reviewer candidate involved in treatment of the patient, which is read from the patient information database based on information for identifying the patient, on the display unit; allowing the review applicant to select one or more designated reviewer candidates based on professions of the one or more designated reviewer candidates displayed on the display unit, the one or more designated reviewer candidates being desired to perform review for supporting decision of the treatment plan; selecting, by the processing server, one or more anonymous reviewer candidates extracted randomly based on professions and/or specialties relating to the details to be reviewed from the reviewer information database; and asking the one or more designated reviewer candidates selected by the review applicant and the one or more anonymous reviewer candidates selected by the processing server whether or not to accept a request for cooperation on review.

Further, according to the one embodiment of the present invention, the review applicant is allowed to propose a plurality of options relating to the treatment plan while presenting the details to be reviewed.

Further, the treatment course determination assistance system according to the one embodiment of the present invention further includes a treatment plan past information database having stored therein treatment plan information processed before by the treatment course determination assistance system. Further, according to the one embodiment of the present invention, the processing server is configured to read, based on the input details to be reviewed, similar treatment plan information processed before by the treatment course determination assistance system from the treatment plan past information database and display the similar treatment plan information.

Further, according to the one embodiment of the present invention, the review applicant is capable of selecting the professions and/or the specialties of the one or more anonymous reviewer candidates.

Further, according to the one embodiment of the present invention, the one or more designated reviewer candidates and the one or more anonymous reviewer candidates are allowed to perform review with reference to the patient information database by being assigned with access authorities with respect to the patient information database as reviewers through acceptance of the request for cooperation on review.

Further, according to the one embodiment of the present invention, the reviewers are allowed to for options relating to a plurality of treatment plans proposed by the review applicant together with the details to be reviewed.

Further, according to the one embodiment of the present invention, the reviewers are capable of proposing, as a new option, a treatment plan idea other than options relating to a plurality of treatment plans proposed by the review applicant together with the details to be reviewed.

Further, according to the one embodiment of the present invention, the treatment course determination assistance system is configured to notify, when the new option is proposed, reviewers other than the reviewer that has proposed the new options, of a request for selecting options again.

Further, according to the one embodiment of the present invention, the processing server is capable of aggregating results of review performed by the reviewers and displaying the aggregated results of review on the display unit.

Further, according to the one embodiment of the present invention, the processing server is configured to disclose information on the anonymous reviewers together with the aggregated results of review within a range in which the anonymous reviewers are prevented from being identified.

Further, according to the one embodiment of the present invention, the reviewers are capable of exchanging opinions with each other in an electronic bulletin board.

Further, according to the one embodiment of the present invention, the patient information database and/or the reviewer information database are updated by patient information and/or reviewer information transmitted from outside.

Further, according to the one embodiment of the present invention: the one or more designated reviewer candidates are displayed in a manner identifiable through display of only professions of the one or more designated reviewer candidates; and the review applicant is allowed to select a designated reviewer based on the professions.

According to the present invention, not only the patient but also the primary doctor and medical professionals involved such as a nurse, a nutritionist, a clinical engineer, and a social worker, who are already involved in the treatment, can hear opinions of other medical professionals involved in decision of a treatment plan for the patient, and the patient can decide the treatment plan based on those opinions.

With this, it is possible to improve the quality of the treatment, and allow the medical professionals involved to perform a treatment with a sense of security. Further, it is possible to prevent occurrence of adjustment of a difference in opinions among the medical professionals involved and occurrence of unnecessary treatments, with the result that it is possible to receive a treatment after the patient and his or her family are convinced.

Further, it is possible to receive an opinion easily from an anonymous expert in addition to opinions of the medical professionals involved. Therefore, it is possible to secure fairness and objectivity in decision of the treatment plan and contribute to improvement of the quality of the treatment by using objective opinions of experts other than the medical professionals involved as well.

Further, the reviewer who performs support review for deciding the treatment plan can also access the patient information easily, and express his or her opinion on the treatment plan through exchange of opinions with other medical professionals involved and anonymous reviewers. Therefore, it is possible to secure fairness and objectivity in decision of the treatment plan and contribute to improvement of the quality of the treatment.

Further, both the review applicant and the reviewer can, for example, create, vote on, and express opinions on details to be reviewed while referring to past similar cases. Therefore, it is possible to contribute to improvement of the quality of the treatment through decision of an objective treatment plan while considering past similar cases.

Further, the review applicant can check information on the reviewer along with the disclosure of a review result. However, only information that does not identify an individual such as the age or specialty of the reviewer is disclosed for a randomly extracted anonymous reviewer. Therefore, it is possible to secure fairness and objectivity of the review.

### BRIEF DESCRIPTION OF THE DRAWINGS

FIG. 1 is a diagram for illustrating a configuration of a treatment course determination assistance system according to an embodiment of the present invention.

FIG. 2 is an outline of a flowchart for illustrating processing of the treatment course determination assistance system.

FIG. 3 is a diagram for illustrating details of Step 1 of FIG. 2.

FIG. 4 is a diagram (State 1) of a review application screen (Step 1) for supporting decision of a treatment plan.

FIG. 5 is a diagram (State 2) for illustrating another state of the screen (Step 1) of FIG. 4.

FIG. 6 is a diagram for illustrating details of Step 2 to Step 4 of FIG. 2.

FIG. 7 is a diagram for illustrating details of Step 5 to Step 7 of FIG. 2.

FIG. 8 is a diagram for illustrating a screen for review result. input (Step 5) by the reviewer.

FIG. 9 is a diagram for illustrating a screen for disclosing (Step 6) a review result.

### DESCRIPTION OF EMBODIMENTS

A mode for carrying out a treatment course determination assistance system according to an embodiment of the invention is described below with reference to the accompanying drawings. The treatment course determination assistance system according to the embodiment of the present invention is hereinafter referred to as “support system”.

The support system according to the embodiment of the present invention enables both opinions of a medical professional involved and an anonymous expert to be heard easily and enable those who provided opinions to review a treatment plan for a patient easily and express their opinions easily in decision of the treatment plan.

### Outline of Support System

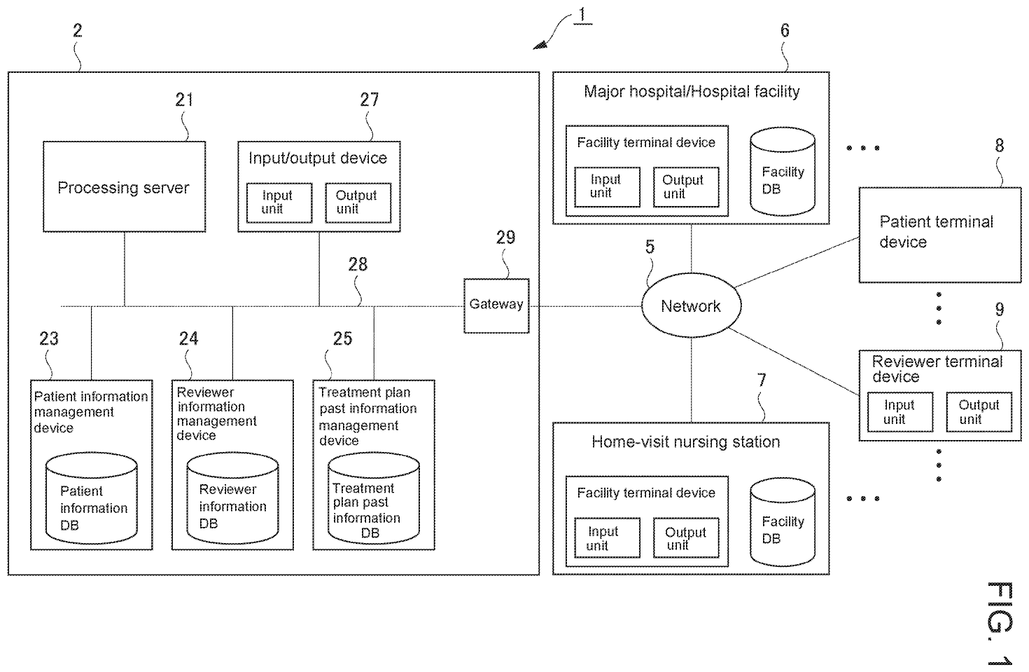

FIG. 1 is a diagram for illustrating a configuration of the support system according to the embodiment of the present invention. As illustrated in FIG. 1, a support system 1 roughly includes: a support center 2 configured to perform processing of managing various kinds of information and supporting decision of a treatment plan; and terminal devices connected to the support center 2 via a network 5, which are to be used for: transmitting the latest information on, for example, patients, medical professionals involved, and. review partners to the support center 2; applying for treatment plan decision support, review and checking results thereof; and reviewing the treatment plan.

The support center 2 configured to perform processing of managing various kinds of information and supporting decision of the treatment plan includes a processing server 21 configured to centrally manage control and calculation relating to the support system 1, a patient information management device 23, a reviewer information management device 24, a treatment plan past information management device 25, and an input/output device 27. The processing server 21 and the management devices (patient information management device 23, reviewer information management device 24, and treatment plan past information management device 25) included in the support center 2 are connected to one another via a local area network (LAN) 28, and are connected to the network 5 via a gateway 29.

The processing server 21, the patient information management device 23, the reviewer information management device 24, the treatment plan past information management device 25, and the input/output device 27 of the support center 2 are formed of computers, and are configured to perform processing by executing programs stored in their storage devices.

The network 5 is formed of an internet line in general, but is not limited thereto. The network 5 may be, for example, a dedicated line as long as the network 5 functions as a communication network.

### Processing Server

The processing server 21 plays a primary role of the support system 2, and is configured to perform the control and calculation relating to the support system 1 after information (patient information, reviewer information, and treatment plan past information) is read from the management devices (patient information management, device 23, reviewer information management device 24, and treatment plan past information management device 25) or after inputs/commands are received from the input/output device 27 or terminal devices outside the support center 2, to thereby support decision of the treatment plan.

### Patient Information Management Device

The patient information management device 23 is configured to manage patient information to be handled by the support system 1, and to, for example, transmit the patient information to the processing server 21. Further, the patient information management device 23 includes a patient information database (patient information DB) that stores patient information such as a name, address, case history, treatment status, and visit history of the patient.

The patient information management device 23 can be, for example, updated to the latest information or corrected via the input/output device 27 in the support center 2. Similarly, the patient information management device 23 is updated to the latest information through input or notification of the latest information from terminal devices outside the support center 2.

Further, in a case where a major hospital/hospital facility 6 or a home-visit nursing station 7 includes a facility DB that stores information on patients including an electronic health record in the facility, the patient information management device 23 may be updated so that the patient information is always updated in synchronization with the facility DB when patient related information in the facility DB is updated.

Further, the patient information management device 23 is configured to read data of the patient information DB in response to an instruction from the processing server 21, and to output the patient information to the processing server 21.

### Reviewer Information Management Device

The reviewer information management device 24 is configured to manage information on the reviewer who performs support review for deciding the treatment plan in the support system 1, and to, for example, transmit the reviewer information to the processing server 21. Further, the reviewer information management device 24 includes a reviewer information database (reviewer information DB) that stores reviewer information such as a name, address, career, specialty, and treatment history of the reviewer.

In the support system 1, the reviewer is, for example, selected from reviewer candidates having a possibility of becoming a reviewer, and thus the reviewer information in the reviewer information DB represents the information described above such as a name and an address of reviewer candidates.

The reviewer information management device 24 can be, for example, updated to the latest information or corrected via the input/output device 27 in the support center 2. Similarly, the reviewer information management device 24 is updated to the latest information through input or notification of the latest information from terminal devices outside the support center 2.

Further, in a case where a major hospital/hospital facility 6 or a home-visit nursing station 7 includes a facility DB that stores information on reviewers (reviewer candidates) in the facility, the reviewer information management, device 24 may be updated so that the reviewer information is always updated in synchronization with the facility DB when reviewer related information in the facility DB is updated.

Further, the reviewer information management device 24 is configured to read data of the reviewer information DB in response to an instruction from the processing server 21, and to output the reviewer information to the processing server 21.

### Treatment Plan Past Information Management Device

The treatment plan past information management device 25 is configured to manage past support information on treatment plan decision support performed using the support system 1, and to, for example, transmit the past support information to the processing server 21. Further, the treatment plan past information management. device 25 includes a treatment plan past information database (treatment plan past information DB) that stores, regarding a case of past treatment plan decision support, treatment plan past information such as the state and case history of another patient relating to the case, details to be reviewed input by the review applicant, an option for review, and the decided treatment plan under a state under which individual information on, for example, another patient is hidden.

The treatment plan past information management device 25 can be, for example, updated to the latest information or corrected via the input/output device 27 in the support center 2.

Further, the treatment plan past information management device 25 is configured to read data of the treatment plan past information DB, and to output the data to the processing server 21 in response to an instruction from the processing server 21.

### Input/Output Device

The input/output device 27 includes at least an input unit and a display unit, and can display (on display unit) information from the processing server 21 and the management devices (patient information management device 23, reviewer information management device 24, and treatment plan past information management device 25), issue various kinds of inputs/commands to the processing server 21, and issue various kinds of inputs/commands to each management device.

Further, when there is erroneous information of the DB in the management devices (patient information management device 23, reviewer information management device 24, and treatment plan past information management device 2), the input/output device 27 can be used to, for example, correct or update the erroneous information.

### Terminal Devices

The terminal devices are used for transmitting the latest information on, for example, patients, medical professionals involved, and review partners to the support center 2; applying for treatment plan decision support review and checking results thereof; and reviewing the treatment plan. The terminal devices are not limited to, for example, a desktop computer, a tablet computer, a laptop computer, or a portable information terminal (PDA) in terms of hardware, and it suffices that the terminal devices include an input/output device capable of enabling input/output of information to/from the support center 2.

Further, the terminal devices correspond to a facility terminal device included in the major hospital/hospital facility 6 involved in treatment of the patient, a facility terminal device included in the home-visit nursing station 7 for providing home-visit nursing services to patients, a patient terminal device 8 possessed by the patient, and a reviewer terminal device 9 possessed by the reviewer who supports review of decision of the treatment plan.

The facility terminals in this case refer to a terminal managed by facilities supported by the support system 1, such as the major hospital/hospital facility 6 and the home-visit nursing station 7. It suffices that the facility terminals are managed by those facilities, and thus the facility terminals also refer to terminals taken out by a primary doctor, a nurse, other medical professionals involved, and the reviewer for a visit outside those facilities, work, or other purposes. Thus, it is obvious that connection to those facilities may be implemented in a wired or wireless manner and connection itself may be permanent or non-permanent connection.

The reviewer terminal 9 refers to a terminal possessed by a person who does not belong to a facility other than the major hospital/hospital facility 6 and the home-visit nursing station 7, and the person is, for example, a member of public support personnel in charge of livelihood protection in an autonomy. Further, the reviewer terminal device 9 also refers to a terminal that does not belong to a facility supported by the support system 1 but is possessed by an expert such as a doctor, a nutritionist, or a clinical engineer involved in the support system 1 as a reviewer.

Further, when the primary doctor or the nurse assigned to the patient possesses a terminal, the reviewer terminal device 9 is a concept that partially overlaps with a facility terminal inside the major hospital/hospital facility 6 and the home-visit nursing station 7.

Further, when the support system 1 is used in the support center 2, the input/output device 27 in the support center 2 is also included in the terminal devices.

### Processing of Support System

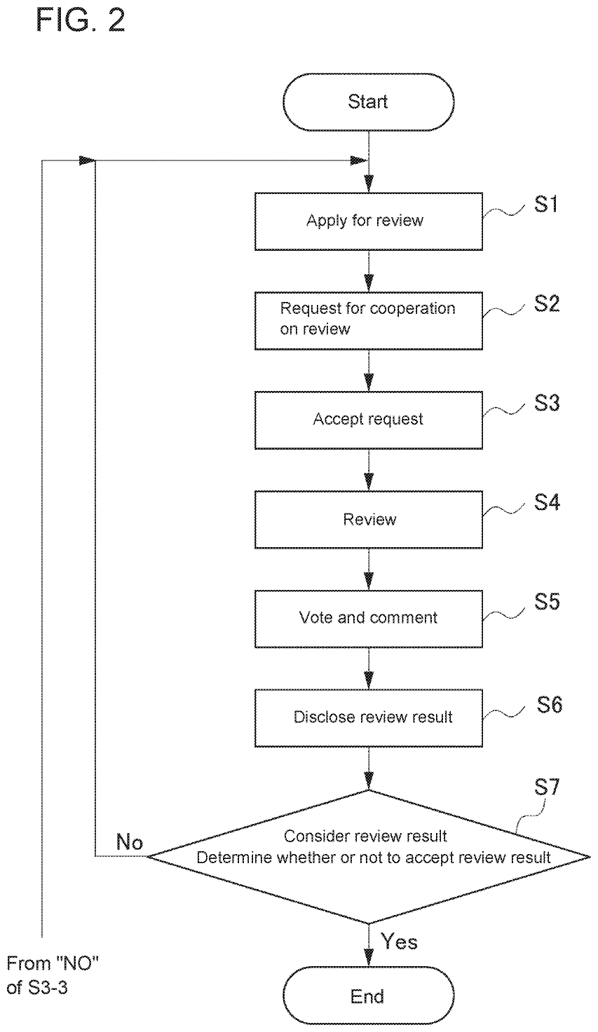

Next, a description is given of processing of supporting decision of a treatment plan in the support system 1 with reference to FIG. 2 to FIG. 9.

### Outline of Processing of Support System

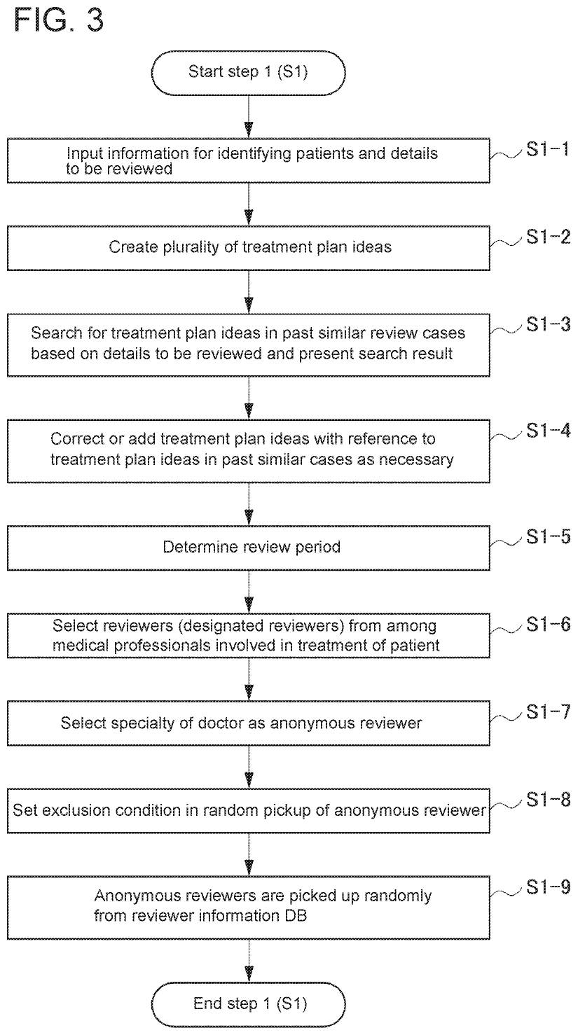

First, an outline of processing of supporting decision of a treatment plan in the support system 1 is described with reference to FIG. 2. FIG. 2 is a flowchart for illustrating an outline of processing of a treatment course determination assistance system. After that, details of the processing are described with reference to FIG. 3 to FIG. 9.

As illustrated in FIG. 2, processing of supporting decision of the treatment plan by the support system 1 is divided broadly into seven steps.

In Step 1 (S1), a review applicant such as a primary doctor, a nurse, a medical professional involved, or a patient applies for review to receive support of decision of the treatment plan, and this review application is performed by inputting a plurality of options relating to details to be reviewed and the treatment plan via an application screen displayed on the terminal devices.

In Step 2 (S2), the support center 2 requests reviewer candidates selected by the support center 2 to cooperate on review for supporting decision of the treatment plan based on the review application input by the review applicant (“review for supporting decision of treatment plan” is hereinafter referred to as “review”)

In Step 3 (S3) the reviewer candidates, who have received requests for cooperation on review, determine whether or not to cooperate on review, and reviewer candidates who are willing to cooperate on review accept a request for cooperation on review. With this, the reviewer candidates become reviewers.

In Step 4 (S4), the reviewers perform review for supporting decision of the treatment plan. In this review, the reviewers are assigned with authorities for access to management apparatus of the support center 2 within a range necessary for review, and perform review with reference to relational information stored in DBs of the management apparatus.

In Step 5 (SS), each reviewer votes for a treatment plan that is determined to be preferable by the reviewer. In this case, the reviewers can vote on options for treatment plans proposed by the review applicant, and can also post comments. Further, when there is no option for a treatment plan that is determined to be preferable by the reviewer, the reviewer can propose another treatment plan instead.

In Step 6 (S6), review results voted for by the reviewers are aggregated and disclosed to the review applicant. At this time, the review applicant can view information on the reviewers that can be disclosed.

In Step 7 (S7), the review applicant determines whether or not to accept the current review for supporting decision of the treatment plan based on the aggregated review results. When the review applicant determines to accept the current review, the support is finished here. On the other hand, when the review applicant determines not to accept the current review, the review applicant applies for another review, returns to Step 1 (S1) as illustrated in FIG. 2, changes details to be reviewed and conditions, and applies for another review, to thereby repeat the same flow.

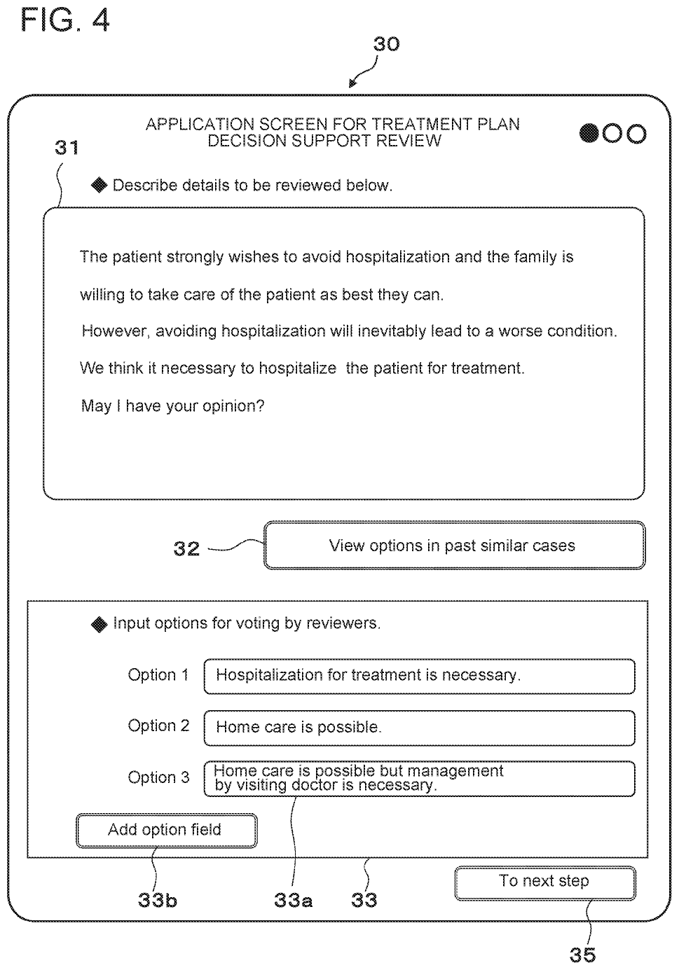

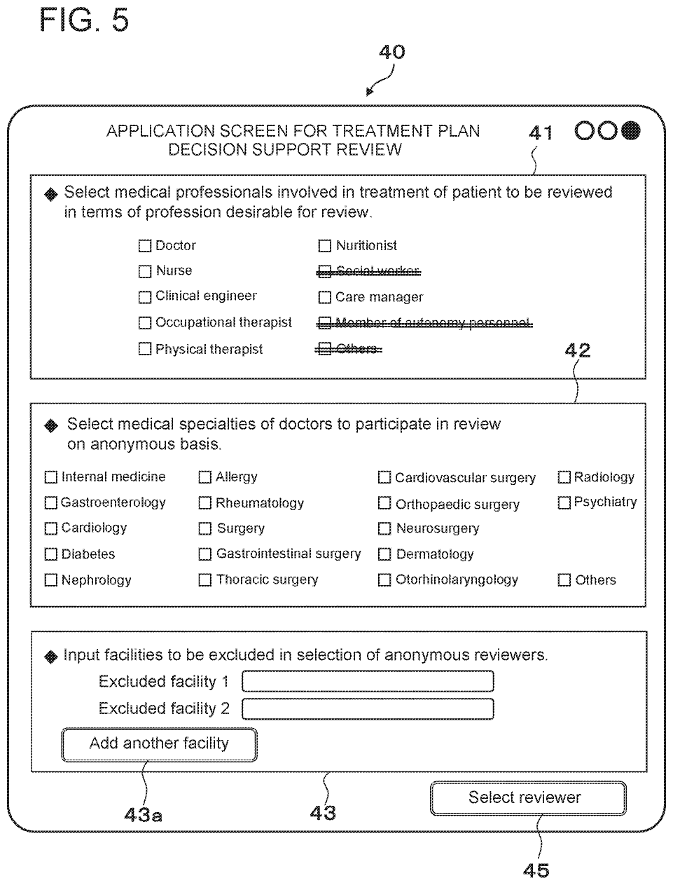

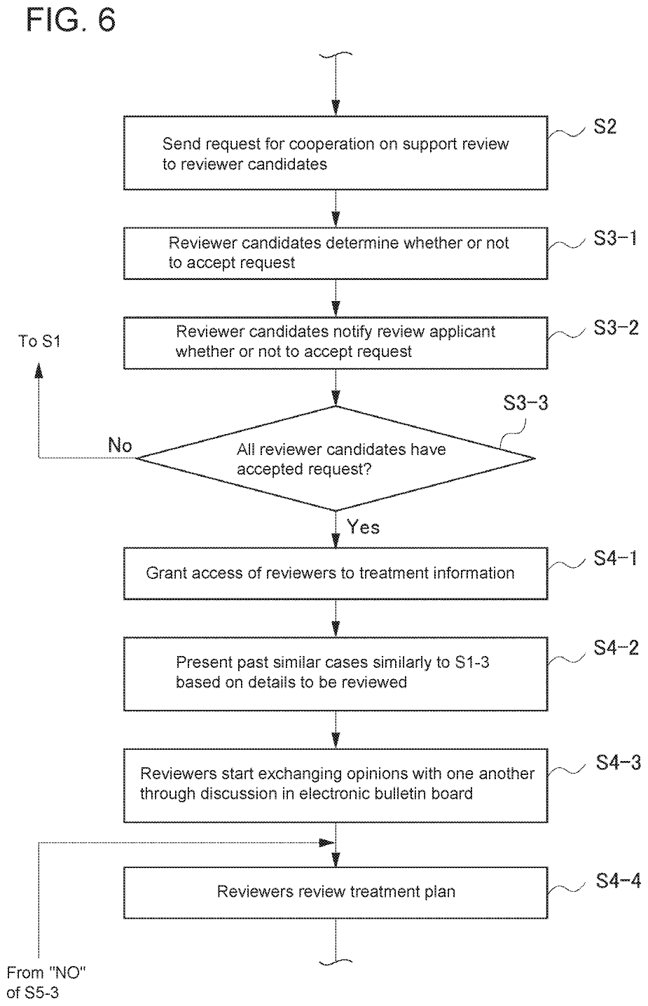

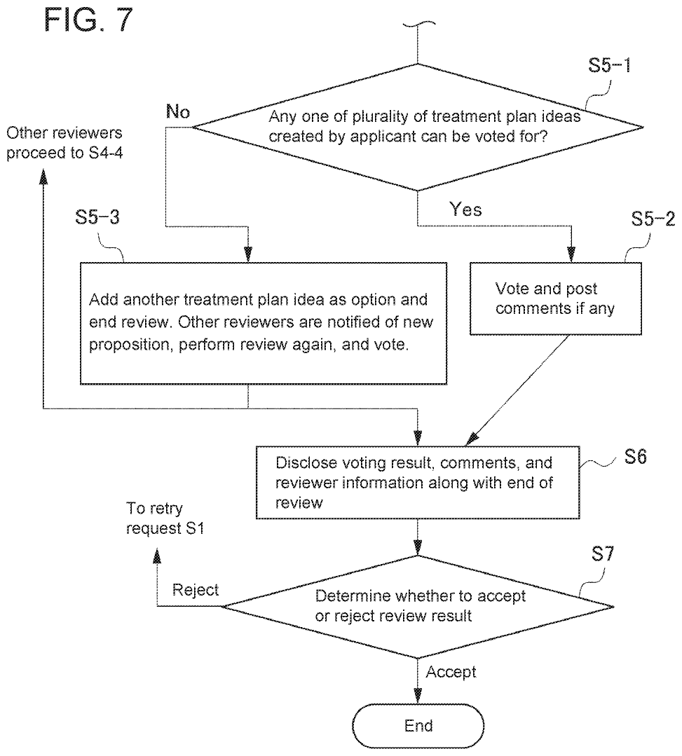

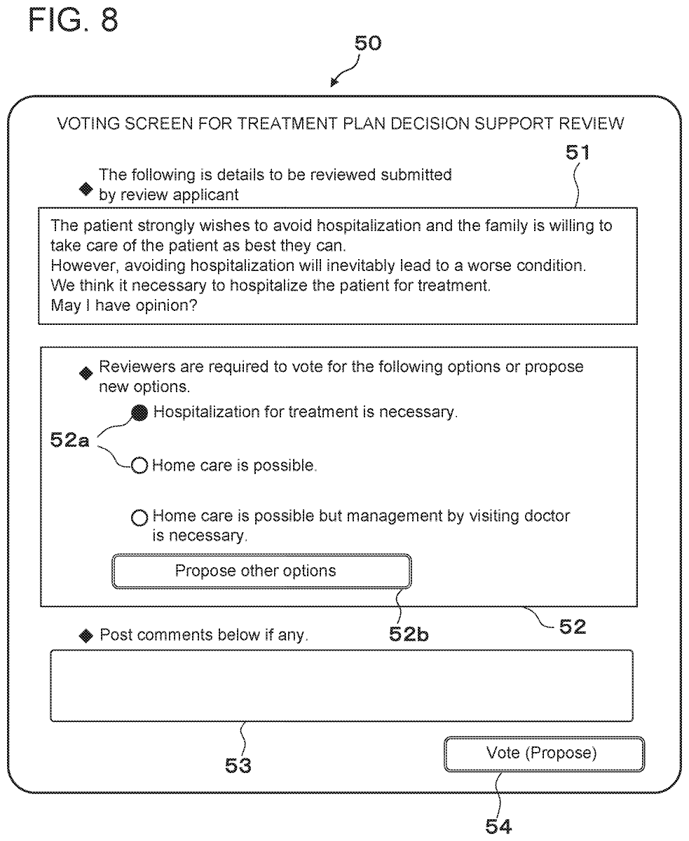

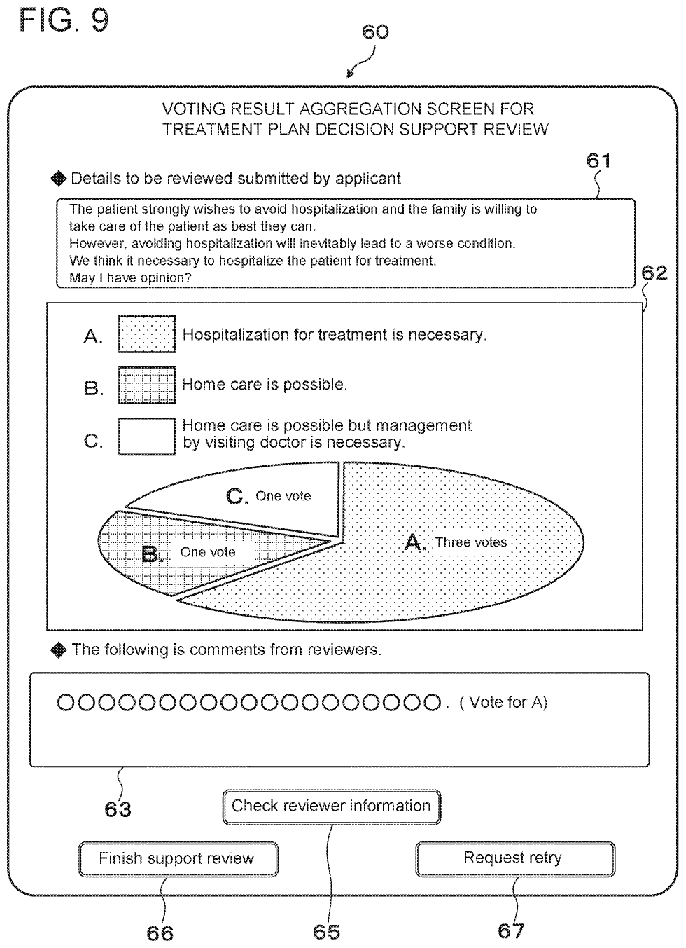

This concludes the description of the outline of the flowchart of the support system 1. Next, a description is given in detail of the processing of supporting decision of the treatment plan by the support system 1 with reference to FIG. 3 to FIG. 9. FIG. 3 is a diagram for illustrating details of Step 1 of FIG. 2. FIG. 4 is a diagram (State 1) of the review application screen (Step 1) for supporting decision of the treatment plan. FIG. 5 is a diagram. (State 2) for illustrating another state of the screen (Step 1) of FIG. 4. FIG. 6 is a diagram for illustrating details of Step 2 to Step 4 of FIG. 2. FIG. 7 is a diagram for illustrating details of Step 5 to Step 7 of FIG. 2. FIG. 8 is a diagram for illustrating a screen for input of a review result (Step 5) by the reviewer. FIG. 9 is a diagram for illustrating a screen for disclosure (Step 6) of a review result.

### Details of Step 1

First, a description is given in detail of Step 1 (S1) of FIG. 2, namely, details of application for review with reference to FIG. 1, FIG. 3, and FIG. 5.

The support system 1 is started by a terminal device accessing the support center 2 via the network 5 and the processing server 21 instructing the display unit of the terminal device that has accessed the support center 2 to display a screen for requesting input of information for identifying a patient, for example, a patient ID.

Further, facilities such as the major hospital/hospital facility 6 and the home-visit nursing station 7 have facility DBs, and when the facility terminal device uses the facility DB to access an electronic health record or a website for patients, a button for starting application for review can be included in those pages on the screen to start application for review.

In this case, the patient to be reviewed is already identified and a page relevant to the patient is already opened. Thus, information for identifying the patient is input in advance, and information for identifying the patient does not need to be input additionally.

To check utilization authority for the support system 1, medical professionals involved such as the primary doctor and the nurse are assigned with a utilization ID and a password for the support system 1 here as review applicants, and this ID and the like can be used to check the utilization authority of the review applicant and to access the support system 1. In addition, the usage history, the viewing history, and the like of the support system 1 are stored in the DB of the treatment plan past information management device 25.

Next, the review applicant is assumed to be the primary doctor, the nurse, the clinical engineer, an occupational therapist, a physical therapist, the nutritionist, the social worker, a care manager, a member of autonomy personnel, and other personnel as a medical professional involved in treatment of the patient.

Further, the review applicant is also assumed to be the patient himself/herself or the family of the patient. However, when the patient or the family is the review applicant, it may be assumed that the patient is prohibited from using the support system 1 alone and is allowed to apply for review with, for example, the primary doctor, only when the ID and the password of, for example, the primary doctor are input in consideration of a case in which the patient and the family have a lack in knowledge of, for example, the medicine, or a case in which the family does not desire the patient to apply for review himself/herself.

Further, when the patient is identified and application for review is started, as illustrated in FIG. 4, the processing server 21 instructs the display unit of the terminal device that has applied for review to display a screen for inputting details to be reviewed as a “application screen for treatment plan decision support review” 30.

Then, the review applicant uses the input unit of the terminal that has applied for review to input details to be reviewed in a review details description field 31. In FIG. 4, for example, the review applicant has input, in the review details description field 31, “The patient strongly wishes to avoid hospitalization and the family is willing to take care of the patient as best they can. However, avoiding hospitalization will inevitably lead to a worse condition. We think it necessary to hospitalize the patient for treatment. May I have your opinion?” (S1-1).

Further, in FIG. 4, the lower part allows options for the treatment plan to be input so that the review applicant can be selected by the reviewer (treatment plan option input field 33) (S1-2). In this embodiment, fields are provided to allow three treatment plans to be input (refer to each input field 33a). Further, an option addition button 33b is provided in preparation for a case in which the three input fields 33a are not sufficient.

In this embodiment, “hospitalization for treatment is necessary”, “home care is possible”, and “home care is possible but management by visiting doctor is necessary” are input as the option 1, the option 2, and the option 3, respectively, in the three input fields 33a.

Further, past similar cases can be referred to for description of options for the treatment plan in the treatment plan option input field 33 (S1-3), and those similar cases can be referred to for description of options. That is, the treatment plan past information management device 25 in the support center 2 includes the treatment plan past information DB that stores information on treatment plans processed an the past.

The processing server 21 instructs, via the similar case view button 32 in the middle of FIG. 4, the treatment plan past information management device 25 to read past similar cases from the treatment plan past information DB based on details input in the review details description field 31, and causes the display unit of the terminal of the review applicant to display the retrieved similar cases so that the review applicant can check the retrieved similar cases (example of display of similar case is not shown). The review applicant can refer to those past similar cases to input, correct, or add options for the treatment plan depending on the desire of the review applicant (S1-4).

Next, the processing transitions to the next input step via a to-next-step button 35 at the bottom of FIG. 4. That is, the review applicant determines and inputs a review period as a deadline for review by a reviewer (S1-5) (relevant screen is not shown).

Next, the processing transitions to an application screen for selecting a reviewer (reviewer candidate), which is a next input step, via a to-next-step button (not shown as well) (“application screen for treatment plan decision support review” 40 of FIG. 5). In general, the selection of reviewers includes a step of selecting reviewers (designated reviewers) from medical professionals involved responsible for the patient and a step of selecting anonymous reviewers (anonymous reviewers) from among people having specialized knowledge other than the medical professionals involved.

The designated reviewers are selected from among the medical professionals involved (S1-6). In this embodiment, as illustrated in a designated reviewer selection field 41 in the upper part of FIG. 5, the designated reviewers can be selected from the doctor, the nurse, the clinical engineer, the occupational therapist, the physical therapist, the nutritionist, the care manager, the member of the autonomy personnel, and other personnel, who are actually involved in treatment of the patient in this case, and the review applicant checks, for selection, the checkboxes of the type of professions of the designated reviewers who are desired to review the treatment plan.

Further, in the example of FIG. 5, strike-throughs are displayed to indicate the fact that social workers, autonomy personnel, and other personnel are not involved in treatment of the patient, and to prohibit selection of those personnel.

To implement selection of those designated reviewers, the processing server 21 instructs the patient information management device 23 to read information on medical professionals involved in treatment of the patient from the patient information DB, and causes the display unit of the terminal of the review applicant to display the read information on medical professionals involved so as to enable the review applicant to check the read information.

Further, in the example of FIG. 5, only the professions of medical professionals involved in treatment of the patient are displayed on the designated reviewer selection field 41, but it may be assumed that not only the professions but also the names of medical professionals actually involved in treatment of the patient are displayed in combination in such a manner as to identify individuals.

Next, an anonymous reviewer is selected randomly from people having specialized knowledge other than medical professionals involved (S1-7).

In this embodiment, as illustrated in an anonymous reviewer selection field 42 in the middle part of FIG. 5, the medical specialty of the doctor necessary for decision of the treatment plan for the patient in this case is selected for selection of anonymous reviewers, and anonymous reviewers are selected randomly from among doctors having the medical specialty. Specifically, the review applicant checks, for selection, the checkboxes of specialties of anonymous reviewers who are desired to review the treatment plan by the review applicant, and anonymous reviewers are selected randomly within the range.

In this embodiment, anonymous reviewers are selected from among people having specialized knowledge other than medical professionals involved by the review applicant selecting the profession and specialty for which the treatment plan is desired to be reviewed and randomly within the range. However, it is also possible for the review applicant to randomly extract the doctor who has a medical specialty similar to that of the primary doctor without the review applicant selecting the profession and specialty of the anonymous reviewer.

In FIG. 5, the doctor is assumed to be an anonymous reviewer, but the anonymous reviewer is not limited to the doctor. Instead, it may be assumed that the review applicant selects professions for a request of cooperation on review, and reviewers are selected randomly in an anonymous manner from those professions and involved in review as anonymous reviewers. For example, it is assumed that, as for supporting decision of the treatment plan relating to nursing, a profession, for example, a care manager, can be selected for cooperation on review as an anonymous reviewer.

Next, an exclusion condition in random extraction (random pickup) of an anonymous reviewer is set (S1-8). A facility that is desired not to be excluded is input to an excluded facility description field 43 in random extraction of an anonymous reviewer at a lower part of FIG. 5. This is to consider a case of reviewing the treatment plan by excluding, for example, past medical professionals that are not recorded in the support system 1 from anonymous reviewers.

Further, in FIG. 5, two input fields are provided in the excluded facility description field 43, but other facilities can be added by using another facility addition field 43a when there are not sufficient input fields.

Further, it is assumed that the exclusion condition can be set not only to facilities but also to various types of exclusion conditions such as the individual name or history, and it is also assumed that the exclusion condition can be set not only to anonymous reviewers but also to designated reviewers selected from medical professionals involved.

The exclusion condition is set, and reviewers are selected via the reviewer selection button 45 at the bottom of FIG. 5. Specifically, designated reviewers are selected from reviewer candidates (designated reviewer candidates) selected in the designated reviewer selection field 41. Further, anonymous reviewers are selected randomly outside the exclusion condition.

To implement selection of those anonymous reviewers, the processing server 21 instructs the reviewer information. management device 24 to read information on people satisfying the condition randomly from the reviewer information DB as anonymous reviewers (anonymous reviewer candidates) (random pickup) (S1-9) This ends Step 1.

### Details of Step 2 and Step 3

Next, a description is given of Step 2 and Step 3 with reference to FIG. 6. In Step 2, the support center 2 requests the reviewer candidates (designated reviewer candidates and anonymous reviewer candidates) selected in Step 1 for cooperation on review.

Specifically, relevant information such as a patient name, a patient information summary, details to be reviewed, and a review period (review deadline) is notified to the reviewer candidates via, for example, an email in response to an instruction from the processing server 21 to ask those reviewer candidates whether or not to accept a request for cooperation on review. The reviewer candidates determine whether or not to accept the request for cooperation on review by accessing the support center 2 (S3-1).

For example, the email may be used only to deliver the fact that there is a request for cooperation on review, and the support center 2 may send only the URL that enables access to the relevant information. Further, application software may be installed into a terminal possessed by a reviewer candidate, and the terminal may be notified of the fact that there is a request for cooperation on review.

Next, the reviewer candidates notify the review applicant whether or not to accept, a request for cooperation on review (S3-2). Specifically, the processing server 21 causes the display unit of the terminal of the review applicant to display a screen asking reviewer candidates having accessed the support center 2 whether or not to accept a request for cooperation on review. The reviewer candidates select an option on the screen to clarify whether or not to accept a request for cooperation on review, and the processing server 21 aggregates the results of selection by all the reviewer candidates to notify the review applicant of the aggregated results.

In this case, the processing server 21 determines whether or not all the reviewer candidates have accepted the request for cooperation on review (S3-3), and when all the reviewer candidates have accepted the request for cooperation on review (YES in S3-3), the reviewer candidates start review as reviewers (end Step 3)

On the other hand, when the processing server 21 does not accept a request for cooperation on review for some reason (No in S3-3), the review applicant is notified of the fact, and returns to Step 1 (S1) to apply for review again.

Instead, it may be assumed that, even when not all the reviewer candidates agree to accept a request for cooperation on review, the review applicant selects an option for starting review.

### Details of Step 4

Next, a description is given of Step 4 with reference to FIG. 6. In Step 4 (S4), the reviewers perform review for supporting decision of the treatment plan.

The reviewers are assigned with access authorities with respect to each management apparatus of the support center 2 within a range necessary for review (access grant: S4-1), and review the treatment plan with reference to the relevant information stored in the DB of each management apparatus.

Further, the reviewers can refer to past similar cases similarly S1-3 of Step 1, and those similar cases can be referred to for review of the treatment plan (S4-2). That is, the processing server 21 instructs the treatment plan past information management device 25 to read past similar cases from the treatment plan past information DB based on the input details to be reviewed, and enables the reviewers to check the retrieved similar cases (example of display of similar cases is not shown).

Further, the reviewers can use an electronic bulletin board that enables the reviewers to exchange opinions with one another along with start of review (S4-3). That is, the reviewers can improve the quality of review by disclosing the own opinion or questioning, for example, experts of other professions in the electronic bulletin board. Further, the electronic bulletin board enables each reviewer to leave a message in the electronic bulletin board when it is convenient for the reviewer.

Further, it may be assumed that the reviewer can view information (e.g., profession or speciality) on other reviewers.

As described above, the reviewers can review the treatment plan (S4-4) by referring to past similar cases (S4-2), exchanging opinions with other reviewers (S4-3), and utilizing their knowledge, to thereby form their views on the treatment plan for the patient within the review period. When the views are formed, the processing proceeds to the next voting step (end Step 4).

### Details of Step 5

Next, a description is given of Step 5 with reference to FIG. 7 and FIG. 8. In Step 5 (S5), each reviewer votes for a treatment plan that is determined to be preferable.

In this embodiment, regarding the vote, as illustrated in FIG. 8, the processing server 21 instructs the display unit of the terminal device of the reviewer to display a screen for inputting options for the treatment plan for which views are formed as a “voting screen for treatment plan decision support review” 50.

Details to be reviewed 51 are illustrated in the upper part of FIG. 8, and an option selection field 52 is arranged in the middle part of FIG. 8 to present three options. The reviewer determines which of the treatment plan ideas created by the review applicant to vote for (S5-1).

When the reviewer determines which of the treatment plan ideas created by the review applicant to vote for (Yes in S5-1), the reviewer selects a check box 52a of any one of the options. In FIG. 8, “hospitalization for treatment is necessary”, which is the first option, is voted for. The reviewer can post a comment using a comment field 53.

Then, the reviewer finishes voting via a vote (proposition) button 54 at the bottom of FIG. 8 to decide on the option to vote for and the comment (S5-2).

It may be assumed that not one but a plurality of options can be set to be voted for.

On the other hand, when the reviewer regards his or her own view on the treatment plan idea as being different from all the treatment plan ideas created by the review applicant (No in S5-1), the reviewer can propose a new treatment plan.

Specifically, the reviewer can open a screen (not shown) for describing a new treatment plan via a new proposition button 52b to propose his or her own treatment plan, and can propose another treatment method. In this case, one option is newly added to the option selection field 52. Then, the reviewer selects the option check box 52a of the treatment plan proposed by the reviewer, and votes for the option (S5-3).

When an option is added as a proposition for a new treatment plan, the other reviewers are notified of the fact that there is a new proposition. In this case, the voting actions of other reviewers who have already finished voting are canceled for the time being, and specifically, the review step of Step 4 (S4) returns to S4-4, with the result that review and voting are performed again in consideration of the treatment plan newly proposed (S5-3).

Further, similar notification, review, and voting are performed again also when other reviewers propose new treatment plans. This ends review of Step 5.

### Details of Step 6 and Step 7

Next, a description is given of Step 6 and Step 7 with reference to FIG. 7 and FIG. 9. Regarding Step 6 (S6), the review results voted for by the reviewers are aggregated and disclosed to the review applicant. Further, in Step 7 (S7), the review applicant determines whether or not to accept the results of the current review for supporting decision of the treatment plan based on the aggregated review results.

In this embodiment, as illustrated in FIG. 9, the voting result of Step 6 (S6) is displayed on the display units of the terminal devices of the review applicant and the reviewers as a “voting result aggregation screen for treatment plan decision support review” 60.

In FIG. 9, details to be reviewed 61 submitted by the review applicant are displayed in the upper part of the screen, and an aggregation result 62 is displayed in the middle part of the screen. In this case, three reviewers have voted for “hospitalization for treatment is necessary”, one reviewer has voted for “home care is possible”, and one reviewer has voted for “home care is possible but management by visiting doctor is necessary”. Thus, it can be understood that a majority of reviewers uphold “hospitalization for treatment is necessary”.

Further, in the bottom part of the screen, comments input by the reviewer can be viewed (comment field 63). It may be assumed that whether a comment is input by a designated reviewer or an anonymous reviewer, the profession or medical specialty of the reviewer, an option voted for by the reviewer, or other items can be displayed. In FIG. 9, it is clarified after the comment of the comment field 63 that the comment is posted by a reviewer who has voted for the option A “hospitalization for treatment is necessary”.

In this embodiment, 5 reviewers review the treatment plan, and one reviewer does not necessarily have one vote. The primary doctor and nurses who communicate with the patient face to face may have a larger number of votes than designated reviewers who have little opportunities to communicate with the patient. Reviewers who viewed documents of DBs of the management apparatus for a longer period of time may have a larger number of votes. In other cases, more experienced reviewers may have a larger number of votes. In those cases, the aggregation result can be displayed by correcting the difference in number of votes.

Further, the review applicant can check information on the reviewers when viewing the review result. In FIG. 9, a reviewer information checking button 65 is arranged in the lower part of the screen, and the review applicant can view information on the reviewers that can be disclosed via this button (reviewer information viewing screen is not shown). The processing server 21 instructs a reviewer information management apparatus 23 to read the reviewer information from the reviewer information DB, and the reviewer information is displayed on the display units of the terminal devices of the review applicant and the reviewer.

In this case, regarding the designated reviewers, who are medical professionals involved in treatment of the patient, information for identifying the individual can be disclosed, but regarding the anonymous reviewers, for example, only the age or medical specialty is disclosed within a range in which the individual is not identified, to thereby secure fairness and objectivity of review.

Similarly, also regarding the designated reviewers, the medical professionals may not be identified, and professions or the like may be used for selection. When there are a plurality of medical professionals involved in a specific profession, information for identifying individuals may not be disclosed.

Next, in Step 7 (S7), the review applicant considers, for example, the disclosed review result and information on the reviewers to examine a subsequent treatment plan. In this case, when the review applicant accepts the review result, the review applicant ends treatment plan decision support review for this case via the support review finish button 66 at the bottom of FIG. 9 (“accept” of S7).

On the other hand, when the review applicant is not satisfied with the review result, or wishes to change conditions to perform review again, the review applicant can request retry via a retry request button 67 at the bottom of FIG. 9. In this case, the review applicant returns to application for review in Step 1 (S1), and enters the procedure for review again.

Further, independently of whether or not the review result is accepted, the processing server 21 instructs the treatment plan past information management device 25 to store the review history in the treatment plan past information DB, and the review history is used for supporting future decision of the treatment plan.

The present invention can be embodied in a large number of forms without departing from the essential characteristics thereof Thus, the above-mentioned embodiment is exclusive for description, and it is to be understood that the present invention is not limited to the above-mentioned embodiment.

1. A treatment course determination assistance system, which is configured to receive opinions from a plurality of reviewers in decision of a treatment plan for a patient, the treatment course determination assistance system comprising:
a processing server configured to perform processing for supporting decision of the treatment plan;
a patient information database having stored therein patient information capable of being read from the processing server;
a reviewer information database having stored reviewer information capable of being read from the processing server; and
an input/output device comprising an input unit configured to input information to the processing server and a display unit configured to output information from the processing server,
wherein the treatment course determination assistance system is configured to perform the steps of:
allowing a review applicant to input information for identifying the patient and details to be reviewed for supporting decision of the treatment plan through use of the input unit;
displaying, by the processing server, a designated reviewer candidate involved in treatment of the patient, which is read from the patient information database based on information for identifying the patient, on the display unit;
allowing the review applicant to select one or more designated reviewer candidates based on professions of the one or more designated reviewer candidates displayed on the display unit, the one or more designated reviewer candidates being desired to perform review for supporting decision of the treatment plan;
selecting, by the processing server, one or more anonymous reviewer candidates extracted randomly based on professions and/or specialties relating to the details to be reviewed from the reviewer information database; and
asking the one or more designated reviewer candidates selected by the review applicant and the one or more anonymous reviewer candidates selected by the processing server whether or not to accept a request for cooperation on review.

2. A treatment course determination assistance system according to claim 1, wherein the review applicant is allowed to propose a plurality of options relating to the treatment plan while presenting the details to be reviewed.

3. A treatment course determination assistance system according to claim 1, further comprising a treatment plan past information database having stored therein treatment plan information processed before by the treatment course determination assistance system,
wherein the processing server is configured to read, based on the input details to be reviewed, similar treatment plan information processed before by the treatment course determination assistance system from the treatment plan past information database and display the similar treatment plan information.

4. A treatment course determination assistance system according to claim 1, wherein the review applicant is capable of selecting the professions and/or the specialties of the one or more anonymous reviewer candidates.

5. A treatment course determination assistance system according to claim 1, wherein the one or more designated reviewer candidates and the one or more anonymous reviewer candidates are allowed to perform review with reference to the patient information database by being assigned with access authorities with respect to the patient information database as reviewers through acceptance of the request for cooperation on review.

6. A treatment course determination assistance system according to claim 5, wherein the reviewers are allowed to vote for options relating to a plurality of treatment plans proposed by the review applicant together with the details to be reviewed.

7. A treatment course determination assistance system according to claim 5, wherein the reviewers are capable of proposing, as a new option, a treatment plan idea other than options relating to a plurality of treatment plans proposed by the review applicant together with the details to be reviewed.

8. A treatment course determination assistance system according to claim 7, wherein the treatment course determination assistance system is configured to notify, when the new option is proposed, reviewers other than the reviewer that has proposed the new option, of a request for selecting options again.

9. A treatment course determination assistance system according to claim 5, wherein the processing server is capable of aggregating results of review performed by the reviewers and displaying the aggregated results of review on the display unit.

10. A treatment course determination assistance system according to claim 9, wherein the processing server is configured to disclose information on the one or more anonymous reviewer candidates who serve as the plurality of reviewers together with the aggregated results of review within a range in which the one or more anonymous reviewer candidates are prevented from being identified.

11. A treatment course determination assistance system according to claim 5, wherein the reviewers are capable of exchanging opinions with each other in an electronic bulletin board.

12. A treatment course determination assistance system according to claim 1, wherein the patient information database and/or the reviewer information database are updated by patient information and/or reviewer information transmitted from outside.

13. A treatment course determination assistance system according to claim 1, wherein:
the one or more designated reviewer candidates are displayed in a manner identifiable through display of only professions of the one or more designated reviewer candidates; and
the review applicant is allowed to select a designated reviewer based on the professions.
